(19) United States
(12) Patent Application Publication
(10) Pub. No.: US 2012/0148595 A1
(43) Pub. Date: Jun. 14, 2012

(54) GABA-LINKED ANTHRACYCLINE-LIPID CONJUGATES

(75) Inventors: Charles S. Swindell, Merion, PA (US); Glenn G. Fegley, Eagleville, PA (US); Hema M. Sundar, West Chester, PA (US); Richard Lawrence, Southold, NY (US)

(73) Assignee: Luitpold Pharmaceuticals, Inc., Shirley, NY (US)

(21) Appl. No.: 13/256,064

(22) PCT Filed: Mar. 11, 2010
(86) PCT No.: PCT/US10/00738

§ 371 Date: Mar. 5, 2012

Related U.S. Application Data
(60) Provisional application No. 61/159,768, filed on Mar. 12, 2009.

(51) Int. Cl. A61K 31/351; A61K 31/664; A61K 38/14; A61K 31/655; A61K 31/475; C07C 271/22; A61K 39/395; A61K 31/7048; A61K 31/519; A61K 31/513; A61P 35/00; A61P 35/02; C07D 309/14; A61K 31/436

(52) U.S. Cl. 424/141.1; 549/417; 514/459; 514/110; 514/19.3; 514/151; 514/283; 514/291; 514/27; 514/249; 514/274; 560/157

(57) ABSTRACT

The present invention relates to GABA-linked anthracycline-lipid conjugates and to methods of using the conjugates to treat cancer. Methods for making the GABA-linked anthracycline lipid conjugates are also provided.

### RELATED APPLICATIONS

This application claims the benefit under 35 U.S.C. §119(e) of U.S. provisional application Ser. No. 61/159,768, filed on Mar. 12, 2009, the entire contents of which are incorporated herein by reference.

### FIELD OF THE INVENTION

The present invention relates to GABA-linked anthracycline-lipid conjugates and to methods of using the conjugates to treat cancer.

### BACKGROUND OF THE INVENTION

Improving drug selectivity for target tissue is an established goal in the medical arts. In general, it is desirable to deliver a drug or a therapeutic agent selectively to its target, so that dosage and, consequently, side effects can be reduced. This is particularly the case for toxic agents such as anticancer agents because achieving therapeutic doses effective for treating the cancer is often limited by the toxic side effects of the anticancer agent on normal, healthy tissue.

Extensive research has been done on the use of fatty acids to improve selectivity of therapeutic agents such as anticancer agents for their target tissues. Fatty acids previously have been conjugated to therapeutic agents to help these agents as conjugates cross the blood brain barrier. DHA (docosahexaenoic acid) is a 22 carbon naturally-occurring, unbranched fatty acid that previously has been shown to be effective, when conjugated to a drug, in crossing the blood brain barrier.

Examples of the conjugation of lipid molecules to therapeutic agents are described in U.S. Pat. Nos. 5,919,815, 5,795,909, 5,580,899, and US patent applications 2003/0065023 and 2002/0177609. The benefits of therapeutic agent-lipid conjugates described in the aforementioned patent documents include: targeting of the therapeutic agent to the tissue of interest, favorably affecting the volume of distribution of the therapeutic agent in the tissue of interest, and reducing toxicity and side effects of the therapeutic agent. Another described benefit of the therapeutic agent-lipid conjugates is that once the lipid is separated from conjugation to the therapeutic agent(s) in vivo, the lipid can be readily metabolized in the body.

The type of lipid molecules employed have included phospholipids, non-naturally occurring branched and unbranched fatty acids, and naturally occurring branched and unbranched fatty acids, ranging from as few as 4 carbon atoms to more than 30 carbon atoms. In one instance, enhanced receptor binding activity was observed (for an adenosine receptor agonist), and it was postulated that the pendant lipid molecule interacted with the phospholipid membrane to act as a distal anchor for the receptor ligand in the membrane micro environment of the receptor. This increase in potency, however, was not observed when the same lipid derivatives of adenosine receptor antagonists were used, and, thus, generalizations were not made possible by those studies.

The exact mechanism by which lipid molecules such as fatty acids help agents conjugated to them cross the blood brain barrier is not yet fully understood. It is believed that the attachment of the lipid molecules to hydrophilic agents renders these agents more hydrophobic (more lipophilic) than unconjugated agents. This increased lipophilicity is believed to help the agents cross the blood brain barrier. Increased lipophilicity has also been suggested as a mechanism for enhancing intestinal uptake of agents into the lymphatic system, thereby enhancing the entry of the conjugate into the brain and also thereby avoiding first-pass metabolism of the conjugate in the liver. Once at or near the tissue target, some have reported, supported by data, that the lipid molecule-agent conjugate must be converted back to the parent agent to become effective.

Lipid molecules terminating in a hydroxyl group (fatty alcohols) and lipid molecules terminating in an amino group (fatty amines) have also been conjugated to drugs via linkers. Examples of linkers used to conjugate fatty alcohols to drugs include, carbonate, carbamate, ester, phosphate, thionocarbamate, guanidine, phosphonate oxime, and thiourea linkages. The linkages of fatty alcohols to therapeutic agents are described in US patent application 2002/0177609. Examples of linkers used to conjugate fatty amines to drugs include carbamate, phosphoramide, phosphonamide, urea, amide, thionocarbamate, thiourea, and guanidine. The linkages of fatty amines to pharmaceutical agents are described in US patent application 2003/0065023.

Various other linkers (e.g., self-immolating linkers) have also been utilized in the synthesis of conjugates of therapeutic agents and ligands or carrier molecules. One example of such linkers is gamma-aminobutyric acid (GABA) (Rosowsky et al., J Med Chem 29, 1872-1876, 1986; Zhang et al., Cancer Research 64, 6707-6715, 2004; U.S. Pat. No. 6,214,345; U.S. Pat. No. 5,652,335; U.S. Pat. No. 5,094,848; US patent application 2006/0105948; and US patent application 2005/0054607). GABA may act as a linker or a spacer between the therapeutic agent and the ligand or carrier molecule.

### SUMMARY OF THE INVENTION

The present invention is based on the unexpected finding that particular GABA-linked anticancer agent-lipid conjugates (i.e., anticancer agents coupled to lipids via GABA) showed superior anti-tumor activity compared to the unconjugated anticancer agent. In particular, linoleyl alcohol-GABA-doxorubicin (LOC-GABA-doxorubicin), oleyl alcohol-GABA-doxorubicin (OOC-GABA-doxorubicin), and DHA-GABA-paclitaxel conjugated at the 2′ position showed superior activity in inhibiting tumor growth than unconjugated doxorubicin and paclitaxel respectively. The anti-tumor activity of LOC-GABA-doxorubicin was studied in three tumor models. LOC-GABA-doxorubicin showed superior anti-tumor activity compared to doxorubicin in the Madison 109 (M109) mouse lung carcinoma model and in the HT29 human carcinoma model but not in the MDA-MB-435 human breast carcinoma model. OOC-GABA-doxorubicin also showed superior anti-tumor activity compared to doxorubicin in the M109 mouse lung carcinoma model. These results are unexpected because other GABA-linked anticancer agent lipid conjugates (e.g., DHA-GABA-paclitaxel conjugated at the 7′ position, and etoposide-GABA-linoleate conjugated at the 2′ or 4′ position) did not show superior anti-tumor activity compared to the unconjugated drug.

There was no correlation between the type of lipid molecule or anticancer agent and the superior anti-tumor activity observed in the conjugated anticancer agent compared to the unconjugated anticancer agent. These findings are further illustrated in the Examples below.

Based on the teachings of the prior art, one of ordinary skill in the art would not be able to predict which GABA-linked anticancer agent-lipid conjugates will show improved anti-tumor activity compared to the unconjugated anticancer agent.

It is, however, expected that anthracycline agents with structural and functional similarities to doxorubicin (e.g., daunomycin, epirubicin and idarubicin) would show similar superior anti-tumor activity compared to the unconjugated anthracycline.

According to one aspect of the invention, a compound having a structure

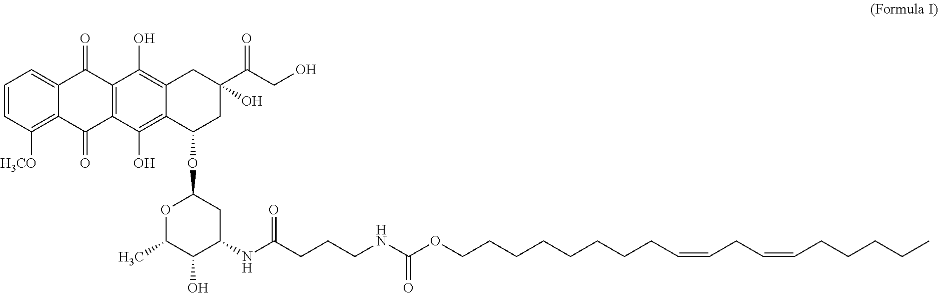

is provided.

According to another aspect of the invention, a pharmaceutical composition is provided. The pharmaceutical composition comprises the compound of Formula I and a pharmaceutically acceptable carrier. The pharmaceutical composition may further comprise an agent other than the compound of Formula I. In some embodiments, the agent is an anticancer agent. Examples of anticancer agents include but are not limited to cyclophosphamide, paclitaxel, taxotere, bleomycin, dacarbazine, vincristine, vinblastine, rapamycin, monoclonal antibodies, etoposide, methotrexate and fluorouracil.

According to another aspect of the invention, a pharmaceutical composition is provided. The pharmaceutical composition comprises the compound of Formula I, 10% Cremophor® EL-P, 10% ethanol, and 80% saline. The pharmaceutical composition may further comprise an agent other than the compound of Formula I. In some embodiments, the agent is an anticancer agent.

In one embodiment, a method for treating a subject having a cancer is provided. The method comprises administering to the subject an effective amount of a pharmaceutical composition of the compound of Formula I to treat the cancer. Examples of cancers that may be treated by the pharmaceutical compositions of the invention are listed below. In some important embodiments the cancer is leukemia (e.g., acute lymphocytic leukemia and chronic lymphocytic leukemia), Hodgkin's lymphoma, multiple myeloma, lung cancer, head and neck cancer, thyroid cancer, endometrial cancer, bladder cancer, ovarian cancer, cervical cancer, breast cancer, stomach cancer, testicular cancer, prostate cancer, soft tissue sarcoma, AIDS-related Kaposi's sarcoma, or Wilms' tumor.

According to another aspect of the invention, a compound having a structure:

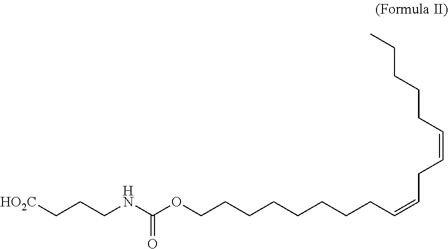

is provided.

According to another aspect of the invention, a compound having a structure:

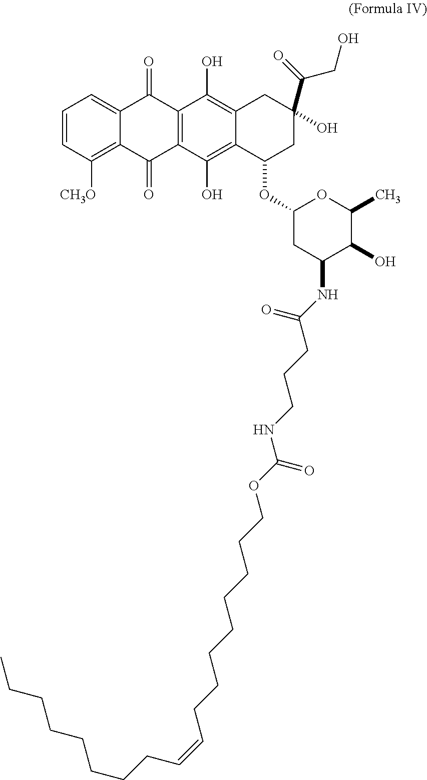

is provided.

According to another aspect of the invention, a pharmaceutical composition is provided. The pharmaceutical composition comprises the compound of Formula IV and a pharmaceutically acceptable carrier. The pharmaceutical composition may further comprise an agent other than the compound of Formula IV. In some embodiments, the agent is an anticancer agent. Examples of anticancer agents include but are not limited to cyclophosphamide, paclitaxel, taxotere, bleomycin, dacarbazine, vincristine, vinblastine, rapamycin, monoclonal antibodies, etoposide, methotrexate and fluorouracil.

According to another aspect of the invention, a pharmaceutical composition is provided. The pharmaceutical composition comprises the compound of Formula IV, 10% Cremophor® EL-P, 10% ethanol, and 80% saline. The pharmaceutical composition may further comprise an agent other than the compound of Formula IV. In some embodiments, the agent is an anticancer agent.

In one embodiment, a method for treating a subject having a cancer is provided. The method comprises administering to the subject an effective amount of a pharmaceutical composition of the compound of Formula IV to treat the cancer. Examples of cancers that may be treated by the pharmaceutical compositions of the invention are listed below. In some important embodiments the cancer is leukemia (e.g., acute lymphocytic leukemia and chronic lymphocytic leukemia), Hodgkin's lymphoma, multiple myeloma, lung cancer, head and neck cancer, thyroid cancer, endometrial cancer, bladder cancer, ovarian cancer, cervical cancer, breast cancer, stomach cancer, testicular cancer, prostate cancer, soft tissue sarcoma, AIDS-related Kaposi's sarcoma, or Wilms' tumor.

According to another aspect of the invention, a compound having a structure:

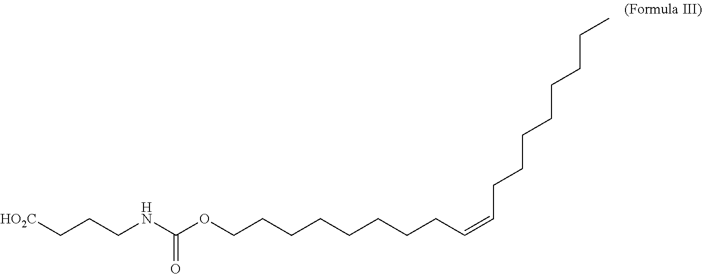

is provided.

These and other aspects of the invention, as well as various advantages and utilities, will be more apparent with reference to the detailed description of the invention. Each aspect of the invention can encompass various embodiments, as will be understood.

### BRIEF DESCRIPTION OF THE DRAWINGS

FIG. 1 is a graph showing the effect of indicated treatments on tumor volume as a function of time. Data show that LOC-GABA-doxorubicin is more active than doxorubicin in the M109 Model.

FIG. 2 is a histogram showing the stability of LOC-GABA-doxorubicin in Cremophor:ethanol:saline (10:10:80) at 10 mg/mL at pH 7.5 mixed with mouse plasma.

### DETAILED DESCRIPTION OF THE INVENTION

The invention described herein relates to GABA-linked anthracycline-lipid conjugates and methods of using the conjugates in the treatment of cancer. The invention provides compositions of matter. The invention also encompasses methods of preparing and conjugating anthracyclines (e.g., doxorubicin, daunomycin, epirubicin, idarubicin and/or any derivatives thereof) to lipids (e.g. C₈ to C₂₂ fatty alcohols such as stearyl alcohol, oleyl alcohol, linoleyl alcohol and docosahexaenoyl alcohol). Examples of methods and processes of making the compositions are described herein, although one of ordinary skill in the art will recognize that there may be other possible synthetic methods.

Anthracyclines are a class of chemotherapeutic agents that inhibit DNA and RNA synthesis by intercalating between base pairs of the DNA/RNA strand, thus preventing the replication of rapidly-growing cancer cells. They also create iron-mediated free oxygen radicals that damage the DNA and cell membranes. Examples of anthracyclines include doxrorubicin, daunomycin, epirubicin, and idarubicin. In some preferred embodiments, the anthracycline is doxrorubicin.

Doxorubicin was the first anthracycline in clinical use, remains the most widely used anthracycline, and is a mainstay of cancer chemotherapy. Many tumors, both solid and hematogenous, respond to doxorubicin. Unfortunately, it has a number of serious toxicities, including myelosuppression, nausea, vomiting, diarrhea, mucositis, alopecia, and most seriously acute and chronic cardiac toxicity. The chronic cardiotoxicity is manifested as a dose dependent congestive cardiomyopathy that often leads to congestive heart failure and death. This dangerous toxicity is managed clinically by limiting the cumulative dose of doxorubicin to less than 450 mg/m², in the absence of other risk factors. As the cumulative dose of doxorubicin increases to 550, 600, and 700 mg/m², the incidence of cardiomyopathy increases to 7%, 15%, and 30%, respectively.

The cardiotoxicity is thought to result from high peak concentrations of doxorubicin reached in the mycocardium after intravenous (i.v.) dosing. The mechanism of toxicity is probably due to oxygen radical formation that occurs in the presence of Fe2+ at the peak concentrations in the mitochondrialrich mycocardium. Mitochondria may be the target organelle within the myocytes that are damaged by doxorubicin. Doxorubicin has the following structure:

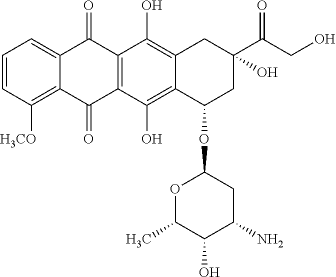

Linoleyl alcohol (9Z,12Z-octadecadien-1-ol) is an 18 carbon atoms, polyunsaturated, a hydrolyzation of linolinic acid, an omega 6 fatty acid. Oleyl alcohol, octadecenol, or cis-9-octadecen-1-ol, is a fatty alcohol and. Its chemical formula is C₁₈H₃₆O or CH₃(CH₂)₇—CH═CH—(CH₂)₈OH. Linoleyl alcohol may be made by converting linoleic acid to linoleyl alcohol using standard methods.

The invention provides compositions of matter. In one aspect of the invention, the composition of matter is compound of Formula I:

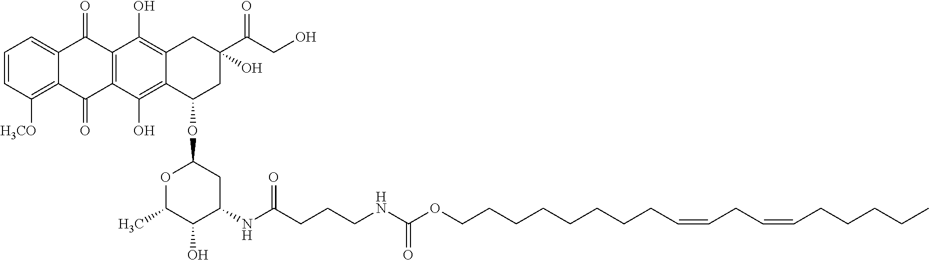

In another aspect of the invention, the composition of matter is compound of Formula IV:

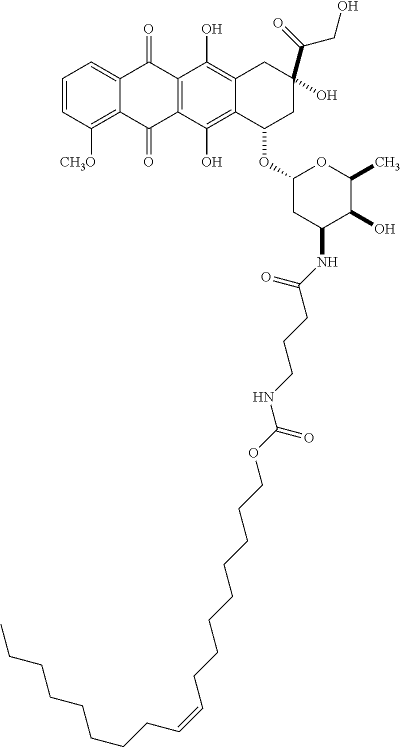

In one aspect of the invention, the compound of the invention (e.g., compound of Formula I or compound of Formula IV) is bound to a label. The label may be a fluorescent label, an enzyme label, a radioactive label, a nuclear magnetic resonance active label, a luminescent label, or a chromophore label. In some embodiments, the label is a fluorine.

In some embodiments, the compound of the invention (e.g., compound of Formula I or compound of Formula IV) is bound to a radioisotope. Some radioisotopes could emit α radiations. Others could emit β radiations. Other radioisotopes could emit γ radiations. Examples of radioisotopes that may be used in this invention include but are not limited to ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I or ⁶⁷Cu, ¹²⁵I, ¹²³I or ⁷⁷Br.

The invention also provides pharmaceutical compositions comprising a compound of the invention (e.g., compound of Formula I or compound of Formula IV). The pharmaceutical composition comprises the compound of the invention (e.g., compound of Formula I or compound of Formula IV) in a pharmaceutically acceptable carrier or diluent.

The term “pharmaceutically acceptable carrier” as used herein refers to compounds suitable for use in contact with recipient subjects, preferably mammals, and more preferably humans, and having a toxicity, irritation, or allergic response commensurate with a reasonable benefit/risk ratio, and effective for their intended use. In some embodiments, the pharmaceutically acceptable carrier is an aqueous solution (e.g., saline).

The pharmaceutical compositions also can contain other components useful in formulating pharmaceutical preparations for administration to subjects, preferably humans, including surfactants, solvents, preservatives, diluents, buffering agents and the like, all of which are standard in the pharmaceutical arts.

Suitable surfactants for use with the present invention include non-ionic agents, such as long-chain fatty acids and their water-insoluble derivatives. These include fatty amines such as lauryl acetyl and stearyl amine, glyceryl esters such as the naturally occurring mono-, di- and triglycerides, and fatty acid esters of fatty amines, such as propylene glycol, polyethylene glycol, sorbitan, sucrose and cholesterol. Also useful are compounds that have polyoxyethylene groups added through an ether linkage with an amine group. Compounds that are also useful in the present invention include the polyoxyethylene sorbitan fatty acid esters and polyoxyethylene glycerol and steroidal esters. Some of the preferred surfactants are Cremophor® EL and Cremophor® EL-P, which are polyoxyethylated castor oil surfactants.

It is contemplated that other surfactants may be used to solubilize the compositions described herein. For example, it is contemplated that polysorbate 80, polysorbate 20, sodium laurate, sodium oleate, and sorbitan monooleate may be useful in certain embodiments of the present invention. Anionic surfactants may also be useful in the practice of the present invention. Examples of these include, but are not limited to, sodium cholate, sodium lauryl sulfate, sodium deoxycholate, sodium laurate, sodium oleate, and potassium laurate.

In certain embodiments, dehydrated ethanol may be used as a solvent for the compositions described herein. In other embodiments, glycols such as propylene glycol or polyethylene glycol are within the scope of the invention. Simple complex polyols may also be suitable solvents. Moreover, the use of non-dehydrated amines may also be suitable within the scope of the present invention. It is recognized that the determination of a solvent and its proper concentration to fully solubilize the conjugate, such as compound of Formula I compositions is within the scope of a skilled artisan, and would not require undue experimentation.

Suitable buffering agents include: acetic acid and a salt (1-2% W/V); citric acid and a salt (1-3% W/V); and phosphoric acid and a salt (0.8-2% W/V).

Suitable preservatives include antimicrobial agents, such as, benzalkonium chloride (0.003-0.03% W/V); chlorobutanol (0.3-0.9% W/V); parabens (0.01-0.25% W/V) and thimerosal (0.004-0.02% W/V) and/or suitable antioxidants, such as, ascorbic acid, ascorbyl pamitate, BHA, BHT, hypophosphorous acid, monothioglycerol, potassium metabisulfite, propyl gallate, sodium formaldehyde sulfoxylate, sodium metabisulfite, sodium bisulfite, sodium thiosulfate, sulfur dioxide, tocopherol and/or tocopherols excipient.

In some embodiments, the compound of Formula I is provided in the form of a pharmaceutically acceptable salt. By “pharmaceutically acceptable salt” is meant those salts which are, within the scope of sound medical judgment, suitable for use in contact with the tissues of a subject without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 66:1 (1977). The salts may be prepared during the final isolation and purification of the compounds of the invention or separately. The salts may be prepared by reacting a free base function with a suitable acid to form the salt (acid addition salts) or by reacting a carboxylic acid-containing moiety with a suitable base (base addition salts). Examples of suitable bases include hydroxide, carbonate, or bicarbonate of a pharmaceutically acceptable metal cation or with ammonia or organic primary, secondary, or tertiary amine.

Representative acid addition salts include, but are not limited to acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate, nicotinate, 2-Naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids that can be employed to form pharmaceutically acceptable acid addition salts include inorganic acids, such as hydrochloric acid, hydrobromic acid, sulfuric acid, and phosphoric acid, and organic acids, such as oxalic acid, maleic acid, succinic acid, citric acid.

Representative pharmaceutically acceptable basic addition salts include, but are not limited to, cations based on alkali metals or alkaline earth metals, such as lithium, sodium, potassium, calcium, magnesium, and aluminum, and the like, and nontoxic quaternary ammonia and amine cations including ammonium, tetramethylammonium, tetraethyl-ammonium, methylamine, dimethylamine, trimethylamine ethylamine, diethylamine, triethylamine, and the like. Other representative organic amines useful for the formation of base addition salts include ethylenediamine, ethanolamine, diethanolamine, piperidine, piperazine, and the like.

In one aspect, the pharmaceutical compositions comprise compound of Formula I and one or more therapeutic agents. In some preferred embodiments, the therapeutic agent is one or more anticancer agent(s). Examples of anticancer agents that may be used include but are not limited to alkylating agents, an antimetabolites, a type I topoisomerase inhibitors, antimitotic drugs, antibiotics, enzymes, biological response modifiers, differentiation agents, and/or radiosensitizers.

Examples of anticancer agents that may be used in the invention include, but are not limited to actimomycin D, actinomycin D, AD 32V/alrubicin, Adrenocortical suppressant, Adrenocorticosteroids/antagonists, adriamycin, AG3340, AG3433, alkylating agents such as melphalan and cyclophosphamide, Alkyl sulfonates, 5-Azacitidine, 5-azacytidine, Alfa 2b, Aminoglutethimide, Amsacrine (m-AMSA), Anthracenedione, Antiandrogens, Antibiotics, Antiestrogen, Antimetabolites, Antimitotic drugs, Asparaginase, AraC, Azacitidine, azathioprine, bacteriochlorophyll-a, Batimastat, BAY 12-9566, BB2516/Marmistat, BCH-4556, benzoporphyrin derivatives, Biological response modifiers, Bleomycin, BMS-182751/oral platinum, busulfan, Busulfan, bromodeozyuridine, 5-bromodeozyuridine, 2-CdA, Caelyx/liposomal doxorubicin, Campto/Levamisole, Camptosar/Irinotecan, Camptothecin, Carboplatin, carmustaine and poliferposan, Carmustine (BCNU), CDP 845, CDK4 and CDK2 inhibitors, Chlorambucil, chloroethylnitrosoureas cisplatin, CI-994, Cisplatin (cis-DDP), 2-chlorodeoxyadenosine, cladribine, CP-358 (774)/EGFR, CP-609 (754)/RAS oncogene inhibitor, CS-682, 9-AC, Cyclopax/oral paclitaxel, Cyclophosphamide, cytosine arabinoside, cytarabine, Cytarabine HCI, Cytokines, D2163, D4809/Dexifosamide, Dacarbazine, Dactinomycin, daunomycin, Daunorubicin HCI, DepoCyt, desmethylmisonidazole, 2′-deoxycoformycin, dexamethasone, diethylstilbestrol ethynyl estradiol, Differentiation Agents, docetaxel, 2,2′-difluorodeoxycytidine, 2′-difluorodeoxycytidine, docetaxel etoposide, Doxil/liposomal doxorubicin, doxorubicin, Doxorubicin HCI, DX8951f, E7070, E09, Edatrexate, Eniluracil/776C85/5FU enhancer, Enzymes, Epipodophylotoxins, Ergamisol/Levamisole, erythrohydroxynonyladenine (EHNA), estramustine, Estramustine phosphate sodium, Estrogens, Erthropoietin, etanidazole, Ethylenimine, Etoposide (V16-213), Evacet/liposomal doxorubicin, farnesyl transferase inhibitor, Folic Acid analogs, FK 317, Floxuridine, Fludara/Fludarabine, fludarabine phosphate, fluorodeoxyuridine, 5-Fluorouracil (5-FU), Flutamide, fluoxymesterone, fragyline, Furtulon/Doxifluridine, Gallium Nitrite, gemcitabine, G-CSF, Gemzar/Gemcitabine, Glamolec, GM-CSF, hydroxyurea, hematoporphyrin derivatives, Hexamethylmelamine (HMM), HMR 1275/Flavopiridol, hormone analogs, Hormones and antagonists, Hycamtin/Topotecan, hydroxyprogesterone acetate, hydroxyprogesterone caproate, Hydroxyurea (hydroxycarbamide), Idarubicin, Inhibitors, Ifes/Mesnex/Ifosamide, Ifosfamide, 5-iododeoxyuridine, Incel/VX-710, Iodine seeds, interferon-alpha, interferon-β, interfon-γ, Interferon Alfa-2a, Interleukin-2, IL-2, irinotecan, ISI641, L-asparaginase, L-Buthiamine Sulfoxide, leuprolide, Lemonal DP 2202, Leuprolide acetate (LHRH-releasing factor analogue), Leustatin/Cladribine, Lomustine (CCNU), LU 79553/Bis-Naphtalimide, LU 103793/Dolastain, LY264618/Lometexol, Mechlorethamine HCI (nitrogen mustard), medroxyprogesterone acetate, Megestrol, megestrol acetate mitotane, Meglamine GLA, melphalan, 6-Mercaptopurine, Mesna, Metastron/strontium derivative, Metaret/Suramin, metronidazole, Methotrexate (MTX), Methyl glyoxal bis-guanylhydrazone (MGBG), Methylhydrazine derivatives, Methylmelamine, misonidazole, Mitoguazone (methyl-GAG), Mitomycin C, mithramycin, Mitotane (o.p′-DDD), mitoxantrone, Mitoxantrone HCI, MMI270, MMP, MTA/LY231514, naphthalocyanine, naphthalocyanines, nicotinamide, nimorazole, Npe6, Nitrogen mustards, Nitrosourceas, N-methylhydrazine, N-methylhydrazine (MIH), Nonsteroidal antiandrogens, Novantrone/Mitroxantrone, ODN 698, Octreotide, Oral Taxoid, paclitaxel, Paraplatin/Carboplatin, PARP inhibitors, Paxex/Paclitaxel, Pentostatin, PD183805, Pharmarubicin/Epirubicin, pheoboride-a, Photofrin®, Photosensitizers, phthalocyanine, Picibanil/OK-432, pimonidazole, pimonidazole etanidazole, PKC412, Plantinol/cisplatin, Platinium coordination complexes, Plicamycin, poliferposan, Prednisone, prednisone and equivalents, procarbazine, Procarbazine HCI, Progestins, Purine analogs, Pyrimidine analogs, Radiosensitizers, RAS farnesyl transferase inhibitor, retinoic acid derivatives, rubidomycin, RB 6145, RSU 1069, SR4233, Semustine (methyl-CCNU), Semustine Streptozocin, SPU-077/Cisplatin, Substituted urea, TA 2516/Marmistat, tamoxifen, Tamoxifen citrate, Taxane Analog, Taxanes, taxol, Taxol/Paclitaxel, Taxoids, Taxotere, Taxotere/Docetaxel, prodrug of guanine arabinoside, Temodal/Temozolomide, teniposide, Teniposide (VM-26), testosterone propionate, Thioguanine, Thiophosphoramide, 6-Thioguanine, Thiotepa, tin etioporphyrin (SnET2), Thriethylenemelamine, TNP-470, triethylene thiophosphoramide, Tiasofuran, tin etioporphyrin, Topotecan, Triazines, Triethylene, trimetrexate, Tumodex/Ralitrexed, Type I Topoisomerase, UFT (Tegafur/Uracil), valrubicin, Valspodar/PSC833, Vepeside/Etoposide, vinblastine, vinblastine (VLB), Vinblastine sulfate, Vinca alkaloids, vincristine, Vincristine sulfate, vinorelbine, VX-853, Vumon/Teniposide, ZD0101, Xeload/Capecitabine, Yewtaxan/Placlitaxel, YM 116, ZD 0473/Anormed, ZD1839, ZD 9331, or zinc phthalocyanine.

Other examples of anticancer agents may be used in the invention are listed in Table 1.

TABLE 1

| Marketer | Brand Name | Generic Name |
|---|---|---|
| Abbott | TNP 470/AGM 1470 | Fragyline |
| Takeda | TNP 470/AGM 1470 | Fragyline |
| Scotia | Meglamine GLA | Meglamine GLA |
| Medeva | Valstar | Valrubicin |
| Medeva | Valstar | Valrubicin |
| Rhone Poulenc | Gliadel Wafer | Carmustaine + Polifepr Osan |
| Warner Lambert | Undisclosed Cancer (b) | Undisclosed Cancer (b) |
| Bristol Myers | RAS Famesyl Transferase | RAS FamesylTransferase |
| Squib | Inhibitor | Inhibitor |
| Novartis | MMI 270 | MMI 270 |
| Bayer | BAY 12-9566 | BAY 12-9566 |
| Merck | Famesyl Transferase Inhibitor | Famesyl Transferase Inhibitor |
| Pfizer | PFE | MMP |
| Pfizer | PFE | Tyrosine Kinase |
| Lilly | MTA/LY 231514 | MTA/LY 231514 |
| Lilly | LY 264618/Lometexol | Lometexol |
| Scotia | Glamolec | LiGLA (lithium-gamma |
| | | linolenate) |
| Warner Lambert | CI-994 | CI-994 |
| Schering AG | Angiogenesis inhibitor | Angiogenesis Inhibitor |
| Takeda | TNP-470 | n/k |
| Smithkline Beecham | Hycamtin | Topotecan |
| Novartis | PKC 412 | PKC 412 |
| Novartis | Valspodar | PSC 833 |
| Immunex | Novantrone | Mitoxantrone |
| Warner Lambert | Metaret | Suramin |
| Genentech | Anti-VEGF | Anti-VEGF |
| British Biotech | Batimastat | Batimastat (BB94) |
| Eisai | E 7070 | E 7070 |
| Biochem Pharma | BCH-4556 | BCH-4556 |
| Sankyo | CS-682 | CS-682 |
| Agouron | AG2037 | AG2037 |
| IDEC Pharma | 9-AC | 9-AC |
| Agouron | VEGF/b-FGF Inhibitors | VEGF/b-FGF Inhibitors |
| Agouron | AG3340 | AG3340 |
| Vertex | Incel | VX-710 |
| Vertex | VX-853 | VX-853 |
| Zeneca | ZD 0101 (inj) | ZD 0101 |
| Novartis | ISI 641 | ISI 641 |
| Novartis | ODN 698 | ODN 698 |
| Tanube Seiyaku | TA 2516 | Marimistat |
| British Biotech | Marimastat | Marimastat (BB 2516) |
| Celltech | CDP 845 | Aggrecanase Inhibitor |
| Chiroscience | D2163 | D2163 |
| Warner Lambert | PD 183805 | PD 183805 |
| Daiichi | DX8951f | DX8951f |
| Daiichi | Lemonal DP 2202 | Lemonal DP 2202 |
| Fujisawa | FK 317 | FK 317 |
| Chugai | Picibanil | OK-432 |
| Nycomed Amersham | AD 32/valrubicin | Valrubicin |
| Nycomed Amersham | Metastron | Strontium Derivative |
| Schering Plough | Temodal | Temozolomide |
| Schering Plough | Temodal | Temozolonide |
| Liposome | Evacet | Doxorubicin, Liposomal |
| Nycomed Amersham | Yewtaxan | Paclitaxel |
| Bristol Myers Squib | Taxol | Paclitaxel |
| Roche | Xeloda | Capecitabine |
| Roche | Furtulon | Doxifluridine |
| Pharmacia & Upjohn | Adriamycin | Doxorubicin |
| Ivax | Cyclopax | Paclitaxel, Oral |
| Rhone Poulenc | Oral Taxoid | Oral Taxoid |
| AHP | Novantrone | Mitoxantrone |
| Sequus | SPI-077 | Cisplatin, Stealth |
| Hoechst | HMR 1275 | Flavopiridol |
| Pfizer | CP-358, 774 | EGFR |
| Pfizer | CP-609, 754 | RAS Oncogene Inhibitor |
| Bristol Myers Squib | BMS-182751 | Oral Platinum |
| Bristol Myers Squib | UFT (Tegafur/Uracil) | UFT (Tegafur/Uracil) |
| Johnson & Johnson | Ergamisol | Levamisole |
| Glaxo Wellcome | Eniluraci1/776C85 | 5FU Enhancer |
| Johnson & Johnson | Ergamisol | Levamisole |
| Rhone Poulenc | Campto | Irinotecan |
| Pharmacia & Upjohn | Camptosar | Irinotecan |
| Zeneca | Tomudex | Ralitrexed |
| Johnson & Johnson | Leustain | Cladribine |
| Ivax | Paxene | Paclitaxel |
| Sequus | Doxil | Doxorubicin, Liposomal |
| Sequus | Caelyx | Doxorubicin, Liposomal |
| Schering AG | Fludara | Fludarabine |
| Pharmacia & Upjohn | Pharmorubicin | Epirubicin |
| Chiron | DepoCyt | DepoCyt |
| Zeneca | ZD1839 | ZD 1839 |
| BASF | LU 79553 | Bis-Naphtalimide |
| BASF | LU 103793 | Dolastain |
| Shering Plough | Caetyx | Doxorubicin-Liposome |
| Lilly | Gemzar | Gemcitabine |
| Zeneca | ZD 0473/Anormed | ZD 0473/Anormed |
| Yamanouchi | YM 116 | YM 116 |
| Nycomed Amersham | Seeds/I-125 Rapid St | Lodine Seeds |
| Agouron | Cdk4/cdk2 inhibitors | cdk4/cdk2 inhibitors |
| Agouron | PARP inhibitors | PARP Inhibitors |
| Chiroscience | D4809 | Dexifosamide |
| Bristol Myers Squib | UFT (Tegafur/Uracil) | UFT (Tegafur/Uracil) |
| Sankyo | Krestin | Krestin |
| Asta Medica | Ifex/Mesnex | Ifosamide |
| Bristol Meyers Squib | Ifex/Mesnex | Ifosamide |
| Bristol Myers Squib | Vumon | Teniposide |
| Bristol Myers Squib | Paraplatin | Carboplatin |
| Bristol Myers Squib | Plantinol | Cisplatin, Stealth |
| Bristol Myers Squib | Plantinol | Cisplatin |
| Bristol Myers Squib | Vepeside | Etoposide |
| Zeneca | ZD 9331 | ZD 9331 |
| Chugai | Taxotere | Docetaxel |
| Rhone Poulenc | Taxotere | Docetaxel |
| Glaxo Wellcome | Prodrug of guanine | Prodrug of arabinside |
| | arabinside | |
| Bristol Myers Squib | Taxane Analog | Taxane Analog |

Depending on the condition to be treated, the pharmaceutical compositions of the invention can be formulated to include therapeutic agents such as one or more cytokines, lymphokines, growth factors, or other hematopoietic factors which can reduce negative side effects that may arise from, or be associated with, administration of the pharmaceutical composition alone. Cytokines, lymphokines, growth factors, or other hematopoietic factors particularly useful in pharmaceutical compositions of the invention include, but are not limited to, M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF, G-CSF, Meg-CSF, GM-CSF, thrombopoietin, stem cell factor, erythropoietin, angiopoietins, including Ang-1, Ang-2, Ang-4, Ang-Y, and/or the human angiopoietin-like polypeptide, vascular endothelial growth factor (VEGF), angiogenin, bone morphogenic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, BMP receptor IA, BMP receptor IB, brain derived neurotrophic factor, ciliary neutrophic factor, ciliary neutrophic factor receptor a cytokine-induced neutrophil chemotactic factor 1, cytokine-induced neutrophil chemotactic factor 2 alpha, cytokine-induced neutrophil chemotactic factor 2β,β endothelial cell growth factor, endothelin 1, epidermal growth factor, epithelial-derived neutrophil attractant, fibroblast growth factor (FGF) 4, FGF 5, FGF 6, FGF 7, FGF 8, FGF 8b, FGF 8c, FGF 9, FGF 10, FGF acidic, FGF basic, glial cell line-derived neutrophic factor receptor alpha 1, glial cell line-derived neutrophic factor receptor α2, growth related protein, growth related protein α growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor alpha, platelet derived growth factor receptor β, pre-B cell growth stimulating factor, stem cell factor, stem cell factor receptor, transforming growth factor (TGF)α, TGFβ, TGFβ1, TGFβ1.2, TGFβ2, TGF β3, TGF β5, latent TGF β1, TGFβ binding protein I, TGFβ binding protein II, TGFβ binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

The therapeutic index of compositions comprising one or more compounds of the invention may be enhanced by conjugation of the compound(s) with anti-tumor antibodies as previously described (for example, Pietersz and McKinzie, Immunol. Rev. 129:57 (1992); Trail et al., Science 261:212 (1993); Rowlinson-Busza and Epenetos, Curr. Opin. Oncol. 4:1142 (1992)). Tumor directed delivery of compounds of the invention enhances the therapeutic benefit by minimizing potential nonspecific toxicities which can result from radiation treatment or chemotherapy. In one aspect of the invention, the compounds of the invention and radioisotopes or chemotherapeutic agents may be conjugated to the same antibody molecule. The tumor specific antibodies may be administered before, during, or after administration of chemotherapeutic-conjugated antitumor antibody or radioimmunotherapy.

The present invention also provides methods of treating cancer in a subject, comprising administering to the subject an effective amount of a pharmaceutical compound comprising a compound of the invention (e.g., compound of Formula I or compound of Formula IV). Preferably, the methods are employed to treat certain cancers in a subject, such as a mammal. Methods of the invention also are readily adaptable for use in assay systems, e.g., assaying cancer proliferation and properties thereof, as well as identifying compounds that affect cancer progression.

As used herein, a subject includes a mammal, such as a human, non-human primate, cow, rabbit, horse, pig, sheep, goat, dog, cat, or rodent such a rat, mouse or a rabbit. In some embodiments, the subject is a human.

The products and methods of the invention are useful for treating certain cancers. Examples of cancers treatable by compounds of the invention include, but are not limited to solid tumors such as carcinomas and sarcomas. Carcinomas include those cancers derived from epithelial cells which infiltrate (invade) the surrounding tissues and give rise to metastases. Adenocarcinomas are carcinomas derived from glandular tissue, or from tissues which form recognizable glandular structures. Sarcomas are tumors whose cells are embedded in a fibrillar or homogeneous substance like embryonic connective tissue.

The invention also enables treatment of cancers of the myeloid or lymphoid systems, including leukemias, lymphomas, and other cancers that typically do not present as a tumor mass, but are distributed in the vascular or lymphoreticular systems.

Examples of cancers treatable by the present invention include myxoid and round cell carcinoma, biliary tract cancer, choriocarcinoma, gastric cancer, intraepithelial neoplasmas, lymphomas, (e.g., small cell and non-small cell), neuroblastomas, oral cancer, pancreas cancer, and renal cancer, as well as other carcinomas, brain and CNS cancer, connective tissue cancer, esophageal cancer, eye cancer, larynx cancer, oral cavity cancer, skin cancer, and testicular cancer, locally advanced tumors, metastatic cancer, soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer, including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian cancer, primary peritoneal epithelial neoplasms, cervical cancer, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including malignant melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. Methods to potentiate treatment of these and other forms of cancer are embraced by the invention.

The compounds of the invention are administered in effective amounts. An effective amount is a dosage of the therapeutic agent sufficient to provide a medically desirable result. An effective amount means that amount necessary to delay the onset of, inhibit the progression of or halt altogether the onset or progression of the particular condition or disease being treated. In the treatment of cancer, for example, in general, an effective amount will be that amount necessary to inhibit cancer cell replication, reduce cancer cell load, or reduce one or more signs or symptoms of the cancer. When administered to a subject, effective amounts will depend, of course, on the particular cancer being treated; the severity of the cancer; individual patient parameters including age, physical condition, size and weight, concurrent treatment, frequency of treatment, and the mode of administration. These factors are well known to those of ordinary skill in the art and can be addressed with no more than routine experimentation. In some embodiments, it is preferred to use the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

Actual dosage levels of active ingredients in the pharmaceutical compositions of the invention can be varied to obtain an amount of the active compound(s) that is effective to achieve the desired therapeutic response for a particular patient, compositions, and mode of administration. The selected dosage level depends upon the activity of the particular compound, the route of administration, the severity of the condition being treated, the condition, and prior medical history of the patient being treated. However, it is within the skill of the art to start doses of the compound at levels lower than required to achieve the desired therapeutic effort and to gradually increase the dosage until the desired effect is achieved.

The compounds and pharmaceutical compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea.

Compounds of the present invention also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of a compound of this invention include powders, sprays, ointments, and inhalants as described herein. The active compound is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from subcutaneous or intramuscular injection. This result can be accomplished by the use of a liquid suspension of crystalline or amorphous materials with poor water solubility. The rate of absorption of the drug then depends upon its rate of dissolution which, in turn, may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

In such solid dosage forms, the active compound is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In a most preferred embodiment, the excipient or carrier increases uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The active compounds also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the active compounds, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the compounds of the invention. The compound is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl. 5): s. 143-146 (1989) (endothelin-1); Hubbard et al., Annals of Internal Medicine 3:206-212 (1989)(α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated.

Formulations suitable for use with a nebulizer, either jet or ultrasonic, typically comprise a compound of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the inhibitor compound suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the inhibitor and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the compounds and composition of the invention also is contemplated. Nasal delivery allows the passage of the compound or composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the compounds of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of compounds across cell and/or nuclear membranes, compositions of relatively high hybrophobicity are preferred. Compounds can be modified in a manner which increases hydrophobicity, or the compounds can be encapsulated in hydrophobic carriers or solutions which result in increased hydrophobicity.

Generally dosage levels of about 0.1 to about 1000 mg, about 0.5 to about 500 mg, about 1 to about 250 mg, about 1.5 to about 100, and preferably of about 5 to about 20 mg of active compound per kilogram of body weight per day are administered orally or intravenously. If desired, the effective daily dose can be divided into multiple doses for purposes of administration, e.g., two to four separate doses per day.

The invention also encompasses methods of conjugating linoleyl alcohol and doxorubicin using GABA as a linker. Synthetic processes are described herein, although one of skill in the art will recognize that there may be other possible synthetic methods.

The invention is exemplified by the following Example.

### EXAMPLE

### Summary

LOC-GABA-doxorubicin was developed as a product for the treatment of solid and hematologic tumors. LOC-GABA-doxorubicin, whose structure is shown below (Formula I), is a conjugate of the fatty alcohol linoleyl alcohol and doxorubicin that employs GABA as a linker. LOC-GABA-doxorubicin has proven to be superior in murine cancer models to doxorubicin.

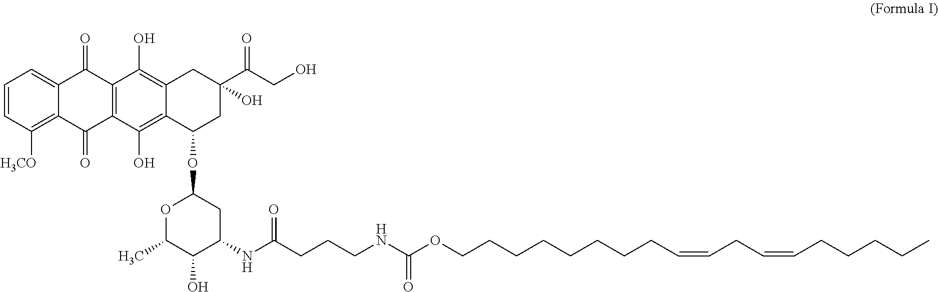

LOC-GABA-doxorubicin has shown activity superior to doxorubicin in the Madison 109 (M109) mouse lung carcinoma model and in the HT29 human colon carcinoma xenograft. It was active, but somewhat less active than doxorubicin, in the MDA-MB-435 human breast carcinoma xenograft model.

LOC-GABA-doxorubicin was synthesized from commercially available doxorubicin through a short three-step sequence. Preliminary development of a Cremophor® EL-P-ethanol formulation for LOC-GABA-doxorubicin has been completed.

### Preclinical Pharmacology

In all of the following in vivo experiments, the parent compound, doxorubicin, was injected intravenously (i.v.) in saline, while the fatty acid conjugates of doxorubicin were injected i.v. in 10% Cremophor® EL-P/10% ethanol/80% saline. The dosing schedule was Q3D×5.

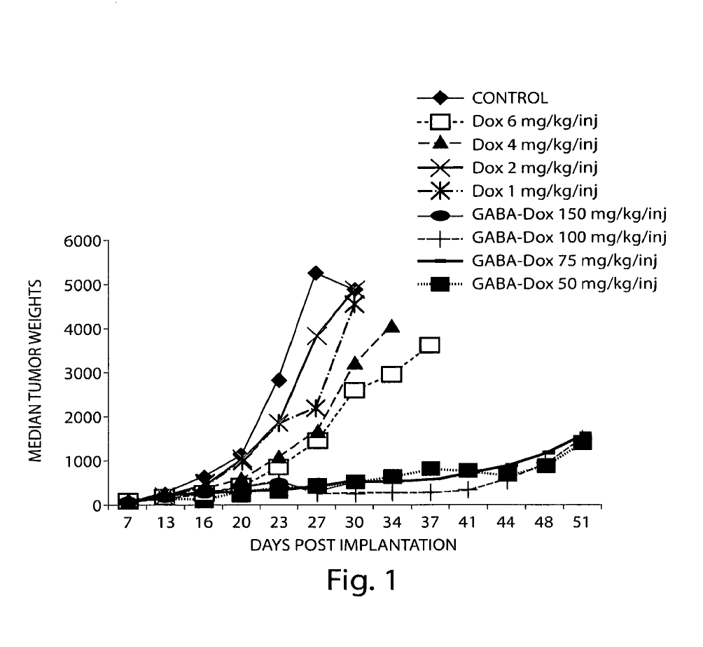

In the Madison 109 (M109) mouse lung carcinoma model, LOC-GABA-doxorubicin suppressed tumor growth much more than did doxorubicin (FIG. 1). Doxorubicin caused no complete responses at any dose and a decrease in tumor growth rate measured as a T-C value of 5.7 days at the Maximum Tolerated Dose (MTD) of 6 mg/kg and of 4.0 at the next lower dose, 4 mg/kg (Table 2). Doxorubicin caused no complete responses at any dose, whereas the LOC-GABA-doxorubicin caused one complete response out of five animals at the 50 mg/kg dose. Doxorubicin again decreased the tumor growth rate by 5.7 days (T-C) at the maximum tolerated dose of 6 mg/kg by only 5-7 days. In contrast, LOC-GABA-doxorubicin decreased tumor growth rate by 25 days T-C and 28 days at the 75 and 50 mg/kg dose responses. T-C in this assay is defined as the time in days for the drug-treated tumors to double their mass three times subtracted from the time in days for the vehicle treated tumors to double their mass three times. In contrast, the LOC-GABA-doxorubicin treated mice had one complete response at 50 mg/kg and T-C values of 25 days for the 75 mg/kg dose and 28 days for the 50 mg/kg dose.

TABLE 2
Data for Doxorubicin and LOC-GABA-doxorubicin in the M109 Model

| | Dose | Drug-related | Complete | Tumor Growth |
|---|---|---|---|---|
| Drug | (mg/kg) | Deaths | Regressions | Delay T-C |
| Doxorubicin | 6 | 015 | 0 | 5.7 |
| | 4 | 0/5 | 0 | 4.0 |
| | 2 | 0/5 | 0 | 2.2 |
| | 1 | 0/5 | 0 | 2.6 |
| LOC-GABA- | 150 | 4/5 | 0 | n/a |
| doxorubicin | 100 | 4/5 | 0 | n/a |
| | 75 | 0/5 | 0 | 25 |
| | 50 | 0/5 | 1 | 28 |

In the nude mouse xenograft model of the HT-29 human colon tumor (Table 3), doxorubicin produced a T-C delay of 15.3 days at the MTD dose of 6 mg/kg, whereas P-367 (LOC-GABA-doxorubicin) produced a T-C delay of 32 days at 50 mg/kg and 22 at 25 mg/kg. Note that the activity of the lowest dose of LOC-GABA-doxorubicin is approximately equivalent to the highest, MTD of doxorubicin itself.

TABLE 3
Data for Doxorubicin and LOC-GABA-doxorubicin in the HT29 model

| | Dose | Drug-related | Complete | Days Delay |
|---|---|---|---|---|
| Drug | (mg/kg) | Deaths | Regressions | T-C |
| Doxorubicin | 6 | 0/10 | 0 | 5.7 |
| | 4 | 0/10 | 0 | 4.0 |
| | 2 | 0/10 | 0 | 2.2 |
| LOC-GABA- | 75 | 6/10 | 0 | 34 |
| doxorubicin | 50 | 0/10 | 0 | 32 |
| | 25 | 0/10 | 0 | 22 |
| | 12.5 | 0/10 | 0 | 5.4 |

In the nude mouse xenograft model of the MDA-MB-435 human breast tumor (Table 4), doxorubicin produced a T-C delay of 14 days at the approximate MTD dose of 6 mg/kg (1/10 drug-related deaths). In the same model, LOC-GABA-doxorubicin produced a T-C delay of 13 days at 50 mg/kg (6/10 drug-related deaths) and 1.5 days at 25 mg/kg (0 drug-related deaths). Thus, in this breast xenograph study, LOC-GABA-doxorubicin is somewhat less efficacious than doxorubicin itself.

TABLE 4
Data for Doxorubicin and LOC-GABA-doxorubicin in the
MDA-MB-435 Model

| | Dose | Drug-related | Complete | Days Delay |
|---|---|---|---|---|
| Drug | (mg/kg) | Deaths | Regressions | T-C |
| Doxorubicin | 6 | 1/10 | 0 | 14 |
| | 4 | 0/5 | 0 | 6.6 |
| | 2 | 0/5 | 0 | 0.0 |
| LOC-GABA- | 75 | 9/10 | 0 | 21 |
| doxorubicin | 50 | 6/10 | 0 | 13 |
| | 25 | 0/10 | 0 | 1.5 |
| | 12.5 | 0/10 | 0 | −2.0 |

### Toxicology

As seen in the Tables 3-5, the MTD of LOC-GABA-doxorubicin is between 50 and 75 mg/kg when given i.v. 5 times on a once every three day schedule. These MTDs are significantly higher than the 6 mg/kg MTD for doxorubicin itself, consistent with the reduced toxicity of LOC-GABA-doxorubicin. Similar results to LOC-GABA-doxorubicin were observed for OOC-GABA-doxorubicin (see Table 5).

TABLE 5
Data for Doxorubicin, LOC-GABA-doxorubicin and OOC-GABA-
Doxorubicin in the M109-Lung Tumor Model

| | Dose | Drug-related | Complete | Days Delay |
|---|---|---|---|---|
| Drug | (mg/kg) | Deaths | Regressions | T-C |
| Doxorubicin | 8 | 0/5 | 0 | 4.6 |
| | 6 | 0/5 | 0 | 0.9 |
| | 4 | 0/5 | 0 | 1.4 |
| | 2 | 0/5 | 0 | 0.0 |
| LOC-GABA- | 80 | 0/5 | 0 | 9.5 |
| doxorubicin | 65 | 0/5 | 0 | 9.3 |
| | 50 | 0/5 | 0 | 5.2 |
| | 35 | 0/5 | 0 | 1.1 |
| OOC-GABA- | 125 | 3/5 | 0 | >6.5 |
| doxorubicin | | | | (Toxic) |
| | 100 | 1/5 | 0 | 10.3 |
| | 75 | 0/5 | 0 | 8.9 |
| | 50 | 0/5 | 0 | 10.0 |

### Chemistry

### A. Sourcing of Doxorubicin Hydrochloride

Doxorubicin hydrochloride available as a dark red crystalline powder was purchased from Hande Tech. Inc. This compound is isolated from Streptomyces peucetius var caesius.

### B. Synthesis of LOC-GABA-Doxorubicin

LOC-GABA-doxorubicin was synthesized through the three-step reaction sequence detailed below.

i. Preparation of N-Hydroxysuccinimidyl Linoleyl Carbonate:

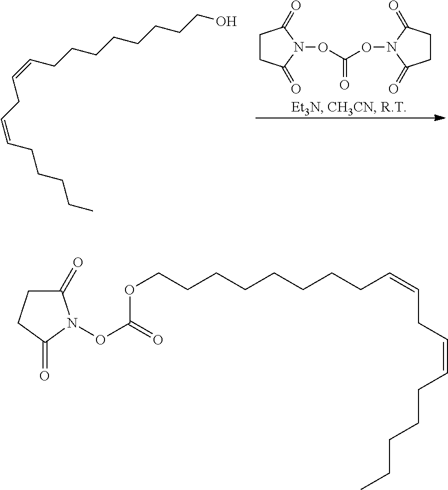

Linoleyl alcohol (5.0 g, 18.91 mmol) was added as a solution in acetonitrile (5 mL) to a suspension of N,N′-disuccinimidylcarbonate (9.7 g, 37.86 mmol) in dry CH₃CN (90 mL), followed by triethylamine (8 mL, 57.39 mmol) and the reaction mixture was stirred at room temperature overnight. The reaction mixture was concentrated under reduced pressure and purified by an ISCO combiflash system using a 110 g column. The product was isolated using gradient elution: 100% hexane-100% ethyl acetate over a period of 30 min.

Yield: 6.2 g, 80.4%.

Chemical Formula: C₂₃H₃₇NO₅

Exact Mass: 407.27

Molecular Weight: 407.54

ii. Synthesis of LOC-GABA (2)

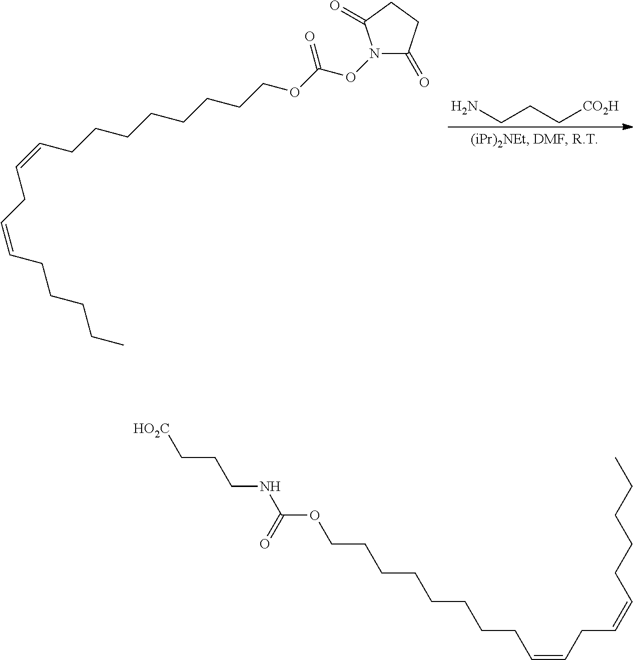

N,N′-diisopropylethylamine (1.3 mL, 7.46 mmol) was added to a suspension of 4-aminobutyric acid (GABA) (525 mg, 5.09 mmol) in dry DMF under argon (10 mL). A solution of N-hydroxysuccinimidyl linoleyl carbonate (1, 2.1 g, 5.15 mmol) in dry DMF (5 mL) was added to the reaction mixture. The reaction mixture was stirred at room temperature for about 18 h. Solvent was removed under high vacuum and the crude reaction mixture was preadsorbed on to silica gel and purified using an ISCO combiflash system with a 35 g column. The product was isolated using gradient elution: 100% hexane-100% ethyl acetate over a period of 30 min and then with 100% ethyl acetate for 15 min. LOC-GABA (2) was isolated as a white solid after evaporation of the solvent and drying at high vacuum for about 18 h. Yield: 1.9 g, 94%.

Chemical Formula: C₂₃H₄₁NO₄

Exact Mass: 395.30

Molecular Weight: 395.58

LC-MS: 434.3 [M+39 (K)], 418.3 [M+23 (Na)], 396.3 (M+1); Exact Mass: 395.3.

1H NMR (360 MHz, CDCl₃): δ 0.89 (t, J=7.2 Hz, 3H), 1.2-1.5 (m, 16H), 1.5-1.75 (m, 2H), 1.84 (m, 2H), 2.02-2.07 (m, 4H), 2.41 (t, J=7.2 Hz, 2H), 2.77 (t, J=7.2 Hz, 2H), 3.15-3.25 (m, 2H), 4.02-4.05 (m, 2H), 4.81 (s, 1H), 5.31-5.40 (m, 4H).

iii. Synthesis of LOC-GABA-Doxorubicin

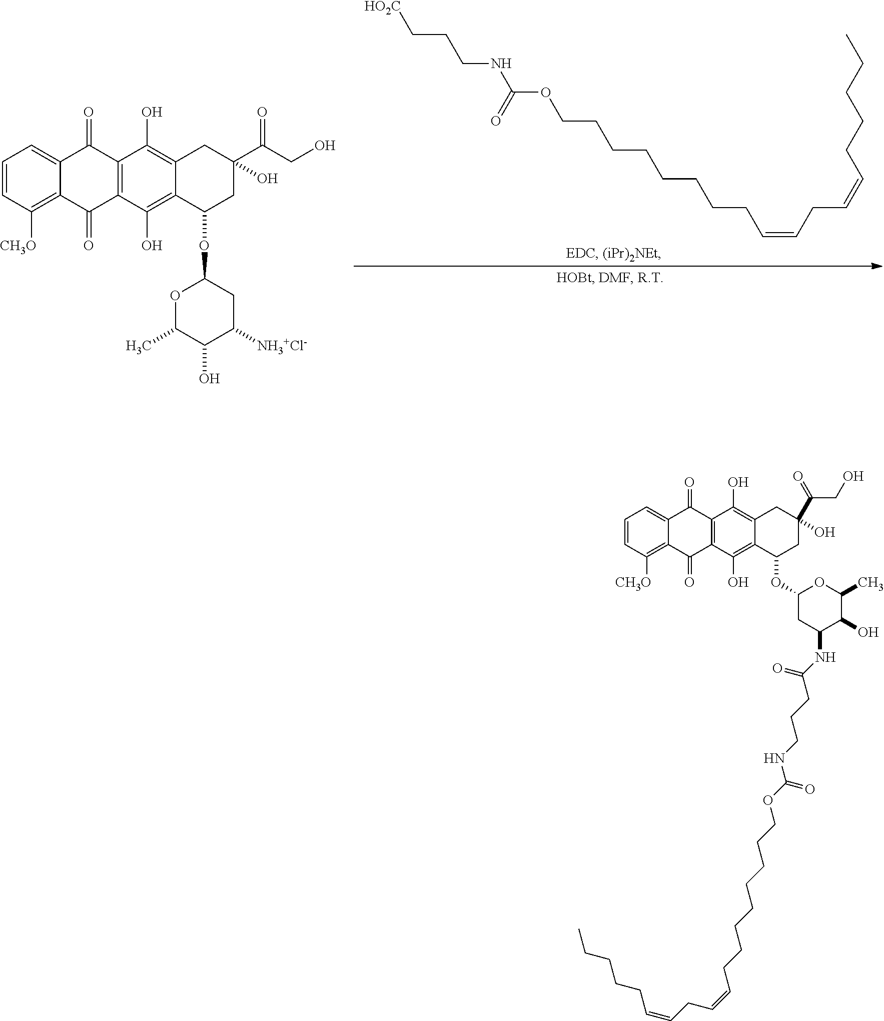

To a suspension of doxorubicin hydrochloride (500 mg, 0.862 mmol) in DMF (10 mL) under argon, N,N′-diisopropylethylamine (450 μL, 2.58 mmol) was added followed by EDC (185 mg, 0.963 mmol), 1-hydroxybenzotriazole (130 mg, 0.962 mmol) and LOC-GABA (2, 380 mg, 0.961 mmol). The reaction mixture was stirred at room temperature for about 18 h. A small amount of bis-acylated product had also formed in addition to the product. Solvent was removed under vacuum and the crude reaction mixture was purified by ISCO combiflash system using a 35 g column. Product was isolated using a gradient elution: 100% CHCl₃-20% CH₃OH in CHCl₃ over 45 min. The product that was isolated contained traces of 1-hydroxybenzotriazole. It was repurified using a gradient elution with 100% ethyl acetate-10% CH₃OH in ethyl acetate over 45 min. Yield: 415 mg, 52%.

C₅₀H₆₈N₂O₁₄

Exact Mass: 920.47

Mol. Wt.: 921.08

LC-MS (negative ion): 919.5 (M−1); Exact Mass: 920.47

1H NMR (360 MHz, CDCl₃): δ 0.89 (t, J=7.2 Hz, 3H), 1.26-1.37 (m, 16H), 1.5-1.6 (m, 2H), 1.74-1.89 (m, 4H), 2.02-2.08 (m, 5H), 2.13-2.20 (m, 4H), 2.35 (d, 1H), 2.77 (t, J=7.2 Hz, 2H), 2.98 (d, J=18 Hz, 1H), 3.25 (d, J=18 Hz, 1H), 3.05-3.28 (m, 3H), 3.72 (s, 1H), 3.99-4.04 (m, 3H), 4.07 (s, 3H), 4.11-4.15 (m, 2H), 4.62 (s, 1 H), 4.77 (s, 2H), 4.88 (t, J=7.2 Hz, 1H), 5.27 (s, 1H), 5.31-5.40 (m, 4H), 5.51 (s, 1 H), 6.15 (d, J=7.2 Hz, 1H), 7.38 (d, J=7.2 Hz, 1H), 7.78 (t, J=7.2 Hz, 1H), 8.02 (d, J=7.2 Hz, 1H).

### C. Synthesis of OOC-GABA-Doxorubicin

OOC-GABA-doxorubicin was synthesized through the three-step reaction sequence detailed below.

i. Preparation of N-Hydroxysuccinimidyl Oleyl Carbonate:

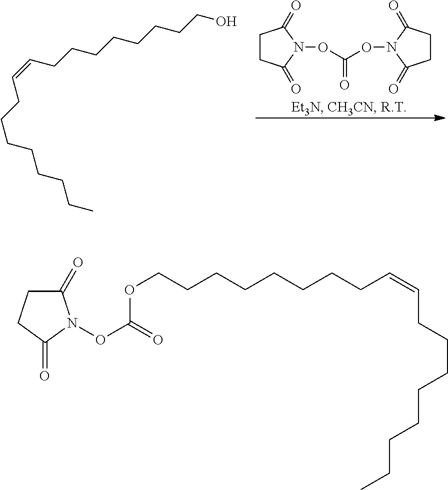

Oleyl alcohol (5.08 g, 18.91 mmol) was added as a solution in acetonitrile (5 ml) to a suspension of N,N′-Disuccinimidylcarbonate (9.70 g, 37.86 mmol) in dry CH₃CN (95 ml), followed by triethylamine (8 ml) and the reaction mixture was stirred at room temperature for 16 hrs. The solvent was removed under vacuum and desired product was purified by silica gel column chromatography using dichloromethane (6.24 g, 80.6%).

Chemical Formula: C₂₃H₃₉NO₅

Exact Mass: 409.28

Molecular Weight: 409.56

1H NMR (400 MHz, CDCl₃): δ 0.81 (t, J=8 Hz, 3H), 1.15-1.34 (m, 22H), 1.64-1.71 (m, 2H), 1.91-1.98 (m, 4H), 2.76 (s, 4H), 4.24 (t, J=8.0 Hz, 2H), 5.23-5.29 (m, 2H).

ii. Synthesis of OOC-GABA

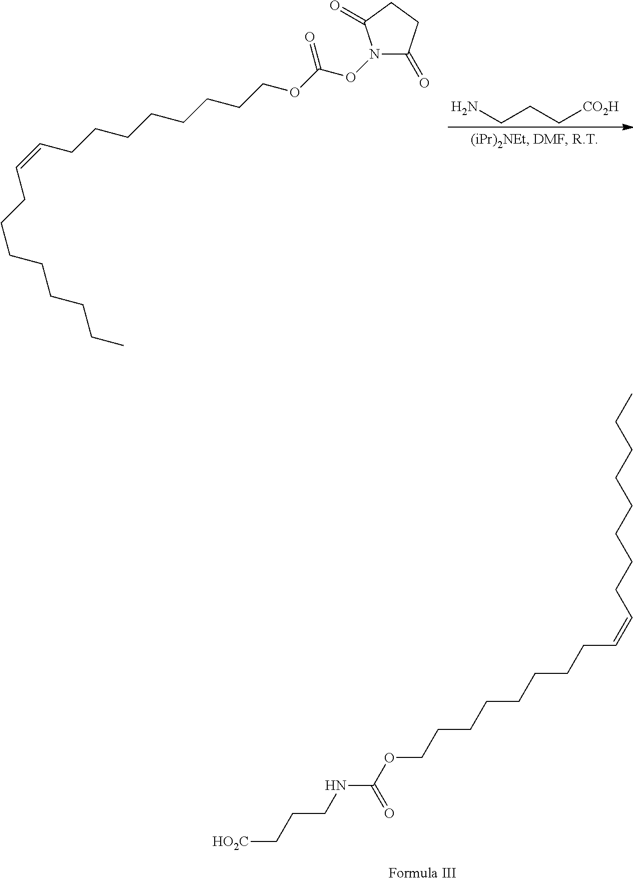

N,N′-Diisopropylethylamine (2.60 g, 14.92 mmol) was added to a suspension of 4-Aminobutyric acid (GABA, 1.05 g, 10.18 mmol) in dry DMF under argon. A solution of N-hydroxysuccinimidyl Oleyl carbonate (4.22 g, 10.30 mmol) in dry DMF (10 ml) was added to the reaction mixture. The reaction mixture was stirred at room temperature for about 21 h. Solvent was removed under high vacuum and the crude residue was purified by silica gel column chromatography using methylene chloride/MeOH (100:0 to 95:5, v/v) to afford white title compound (3.5 g, 87%).

Chemical Formula: C₂₃H₄₃NO₄

Exact Mass: 397.32

Molecular Weight: 397.59

1H NMR (400 MHz, CDCl₃): δ 0.88 (t, J=8 Hz, 3H), 1.25-1.50 (m, 22H), 1.65-1.75 (m, 2H), 1.81-1.88 (m, 2H), 1.99-2.03 (m, 4H), 2.41 (t, J=8.0 Hz, 2H), 3.20-3.30 (m, 2H), 4.00-4.15 (m, 2H), 4.80 (bs, 1H), 5.30-5.39 (m, 2H).

iii. Synthesis of OOC-GABA-Doxorubicin

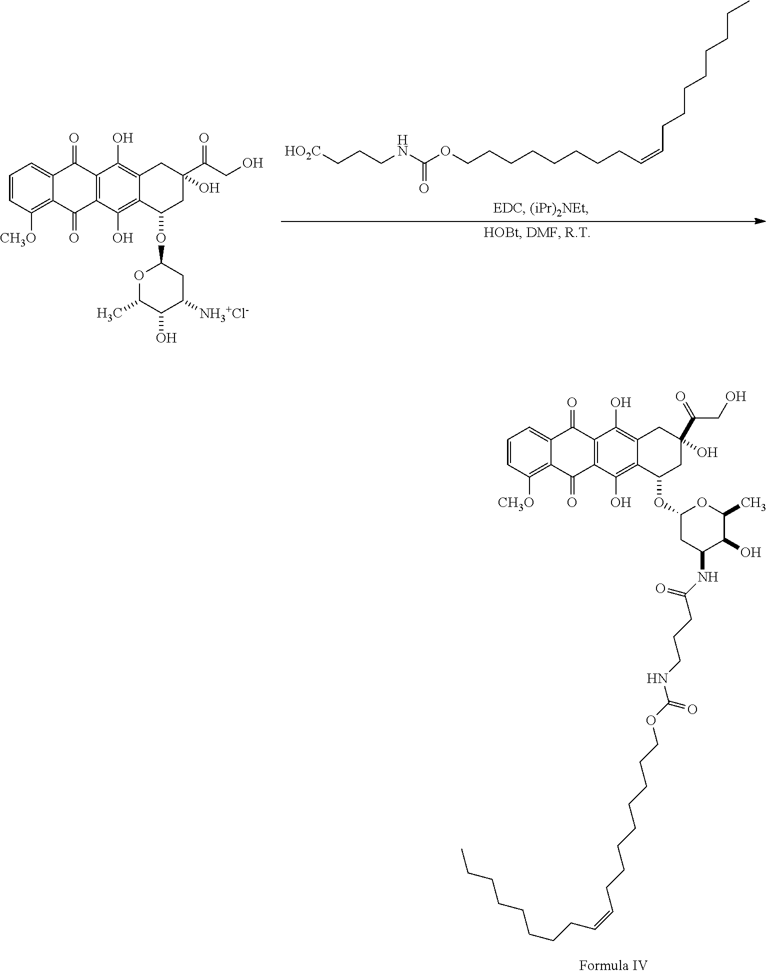

N,N′-Diisopropylethylamine was added to a suspension of Doxorubicin HCl (1.50 g, 2.59 mmol) in dry DMF (30 ml) followed by the addition of EDC:HCl (555 mg, 2.90 mmol), 1-hydroxybenzotriazole (390 mg, 2.89 mmol), and OOC-GABA (1.033 g) in dry DMF (5 ml) under argon. The reaction mixture was stirred at room temperature for 21 h. Solvent was removed under high vacuum and the residue was purified by silica gel column chromatography using EtOAc to 1% MeOH/EtOAc to afford pure red title compound (1 g, purity=99.42%, 0.425 g purity=93%, yield: 1.425 g (1.00 g+0.425 g) 59.6%).

Chemical Formula: C₅₀H₇₀N₂O₁₄

Exact Mass: 922.48

Molecular Weight: 923.10

LC-MS (negative ion): [M−H]⁻=921.5121, [M+H]⁺=923.5213.

1H NMR (400 MHz, CDCl₃): δ 0.88 (t, J=8.0 Hz, 3H), 1.26-1.34 (m, 24H), 1.50-1.95 (m, 7H), 1.96-2.07 (m, 4H), 2.13-2.20 (m, 3H), 2.35 (d, J=16 Hz, 1H), 2.94-3.30 (m, 6H), 3.71 (d, J=8.0 Hz, 1H), 3.99-4.06 (m, 2H), 4.06 (s, 3H), 4.11-4.15 (m, 2H), 4.61 (s, 1H), 4.77 (s, d, J=4 Hz, 2H), 4.90 (m, 1H), 5.26 (s, 1H), 5.31-5.40 (m, 2H), 5.51 (s, 1H), 6.14-6.16 (m, 1H), 7.37 (d, J=8 Hz, 1H), 7.77 (t, J=8.0 Hz, 1H), 8.01 (d, J=8.0 Hz, 1H), 13.19 (m, 1H), 13.94 (s, 1H).

### D. Analytical Methods: HPLC and Mouse Serum Stability Methods

LOC-GABA-doxorubicin in Cremophor® EL-P/ethanol (1:1) was added to mouse serum at a concentration of 50 μmol/L. The final concentration of Cremophor® and ethanol was 1% each. Serum samples added with only ethanol/Cremophor® were used as controls.

Aliquots (100 μL) were taken from the spiked samples at 0, 1, 4, 8 and 24 h time intervals. Proteins were precipitated from the spiked solutions by the addition of 300 μL of acetonitrile. The suspensions were centrifuged and the supernatant transferred to separate microfuge tubes. The precipitate was washed twice with 300 μL of 90% acetonitrile. The wash solutions were combined with the supernatant and evaporated to dryness using a Centrivap Concentrator. The residue was then dissolved in 100 μL of methanol. Vortex mixing and sonication for 30 sec each were used to help dissolve the residue. The solution was then centrifuged at 10,000 rpm for 15 min. A 20 μL aliquot of the supernatant was analyzed by HPLC (Phenomenex column, C18, 250×4.6 mm, 5 u) using the following gradient conditions:

TABLE 6

| Time | 10 mM Ammonium | CH₃CN |
|---|---|---|
| (min) | Formate pH 4.5(%) | (%) |
| 0 | 72 | 28 |
| 5 | 72 | 28 |
| 20 | 0 | 100 |
| 26 | 0 | 100 |
| 30 | 72 | 28 |
| 36 | 72 | 28 |

UV detection: 487 nm; column temperature: 40° C.; flow rate: 1 mL/min. Analytes were quantitated by comparison of peak areas from direct injections of standard solutions with those obtained from samples subjected to the extraction procedure.
The percent recovery of spiked analytes was calculated by comparison of the sample peak areas before incubation (time: 0 h) with those subjected to incubation at 37° C.

### E. Solubility

Table 7 summarizes the solubility of LOC-GABA-doxorubicin in several solvents.

TABLE 7

| Solvent/ | Solubility |
|---|---|
| Excipient | (mg/mL) |
| Ethanol | 252.5 |
| Dimethyl isosorbide (DMI) | 490.5 |
| Cremophor ® EL-P:ethanol: | 16.8 |
| saline (10:10:80) | |

### F. Formulation

LOC-GABA-doxorubicin was formulated in a mixture of Cremophor® EL-P (10%), ethanol (10%), and saline (0.9% NaCl). This was accomplished by dissolving the conjugate in ethanol, adding an equal volume of Cremophor® EL-P, and finally enough saline to result in the final 10%/10%/80% ratio. This formulation was used for the initial in vivo testing of this conjugate.

### G. Stability of LOC-GABA-Doxorubicin in Mouse Serum

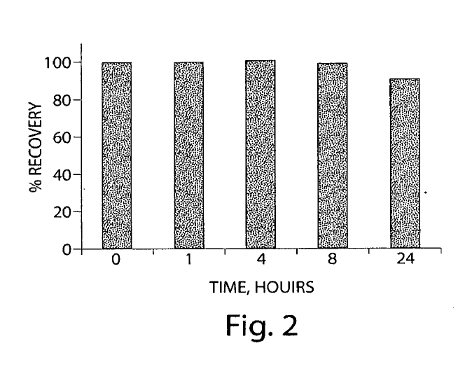

To evaluate the stability of LOC-GABA-doxorubicin in mouse serum, LOC-GABA-DOXORUBICIN was dissolved in Cremophore:ethanol:saline (10:10:80) at 10 mg/mL at pH 7.5 and then mixed with mouse plasma in a ratio of 10 parts formulation to 90 parts plasma and incubated for 0, 1, 4, 8 and 24 hr at 37° C. LOC-GABA-doxorubicin was assayed in the supernatant by HPLC using the previously described method. The results are shown below in FIG. 2.

The stability data shown in FIG. 2 indicate that LOC-GABA-doxorubicin is stable for more than 8 hours in mouse plasma. Indeed, over 90% of the drug is still present at 24 hours.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

We claim:

1. A compound having a structure:

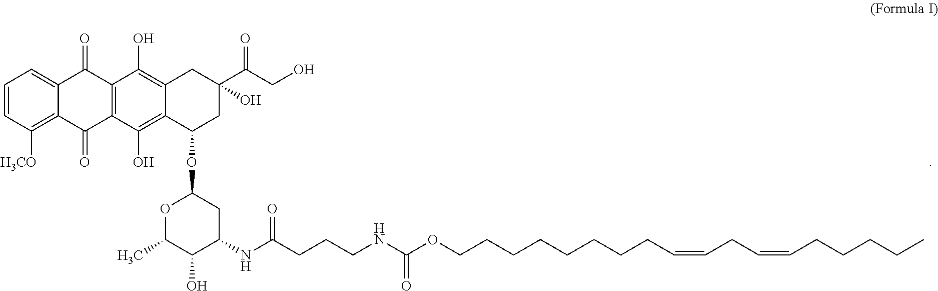

2. A pharmaceutical composition comprising a compound having a structure: 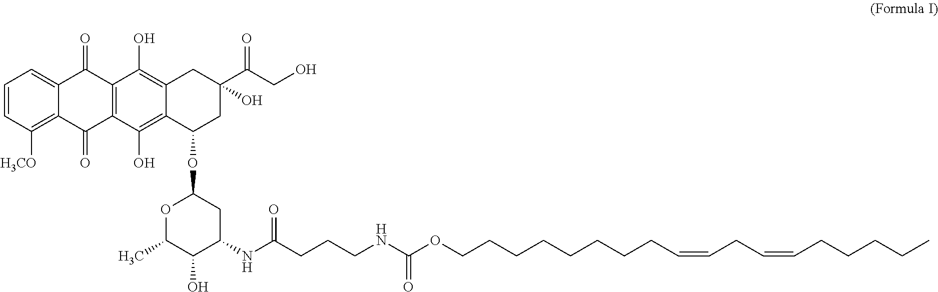
and a pharmaceutically acceptable carrier.

3. The pharmaceutical composition of claim 2 further comprising an agent other than the compound of Formula I.

4. The pharmaceutical composition of claim 3, wherein the agent is an anticancer agent.

5. The pharmaceutical composition of claim 4, wherein the anticancer agent is: cyclophosphamide, paclitaxel, taxotere, bleomycin, dacarbazine, vincristine, vinblastine, rapamycin, monoclonal antibody, etoposide, methotrexate and fluorouracil.

6. A pharmaceutical composition comprising a compound having a structure: 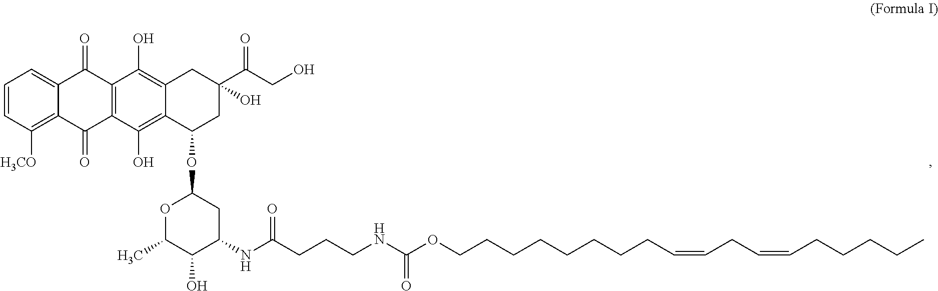
10% Cremophor® EL-P, 10% ethanol, and 80% saline.

7. The pharmaceutical composition of claim 6 further comprising an agent other than the compound of Formula I.

8. The pharmaceutical composition of claim 7, wherein the agent is an anticancer agent.

9. The pharmaceutical composition of claim 4, wherein the anticancer agent is: cyclophosphamide, paclitaxel, taxotere, bleomycin, dacarbazine, vincristine, vinblastine, rapamycin, monoclonal antibody, etoposide, methotrexate and fluorouracil.

10. A method for treating a subject having a cancer comprising
administering to the subject an effective amount of a pharmaceutical composition of claim 2 to treat the cancer.

11. The method of claim 10, wherein the cancer is leukemia (acute lymphocytic leukemia or chronic lymphocytic leukemia), multiple myeloma, lung cancer, head and neck cancer, endometrial cancer, ovarian cancer, cervical cancer, breast cancer, testicular cancer, prostate cancer, or Wilms' tumor.

12. A compound having a structure:

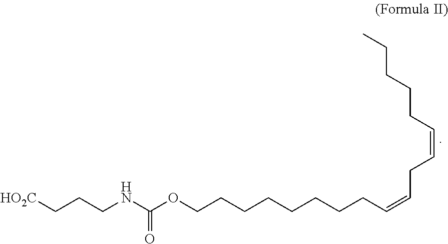

13. A compound having a structure:

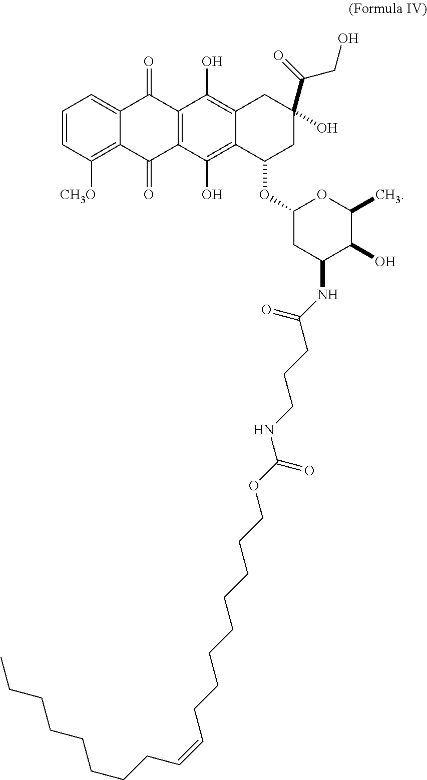

14. A pharmaceutical composition comprising a compound having a structure: 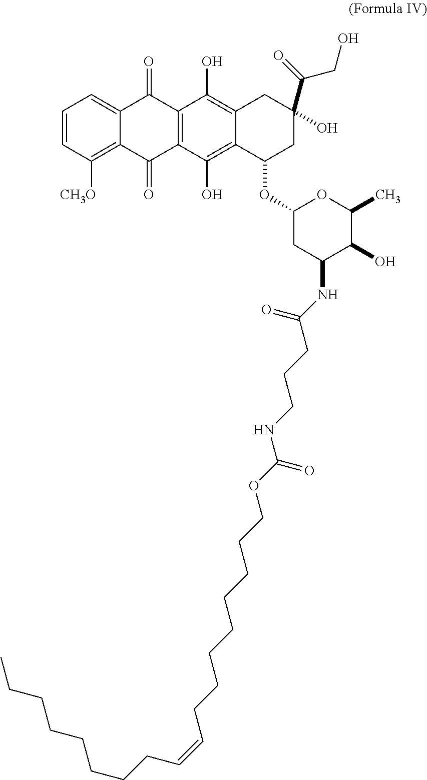
and a pharmaceutically acceptable carrier.

15. The pharmaceutical composition of claim 14 further comprising an agent other than the compound of Formula IV.

16. The pharmaceutical composition of claim 15, wherein the agent is an anticancer agent.

17. The pharmaceutical composition of claim 16, wherein the anticancer agent is: cyclophosphamide, paclitaxel, taxotere, bleomycin, dacarbazine, vincristine, vinblastine, rapamycin, monoclonal antibody, etoposide, methotrexate and fluorouracil.

18. A pharmaceutical composition comprising a compound having a structure: 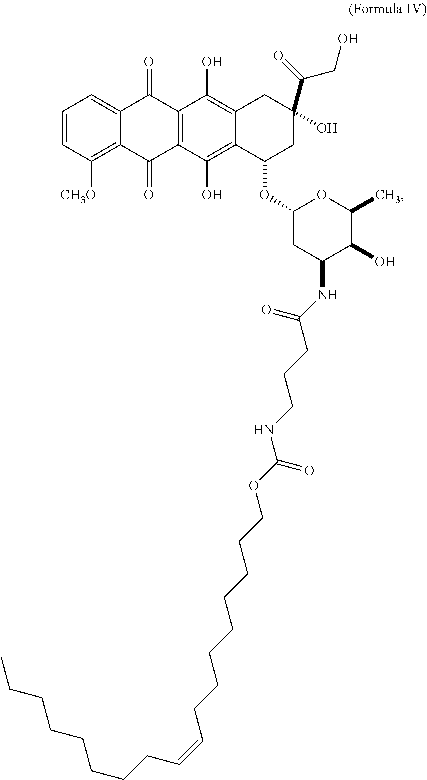
10% Cremophor® EL-P, 10% ethanol, and 80% saline.

19. The pharmaceutical composition of claim 18 further comprising an agent other than the compound of Formula IV.

20. The pharmaceutical composition of claim 19, wherein the agent is an anticancer agent.

21. The pharmaceutical composition of claim 16, wherein the anticancer agent is: cyclophosphamide, paclitaxel, taxotere, bleomycin, dacarbazine, vincristine, vinblastine, rapamycin, monoclonal antibody, etoposide, methotrexate and fluorouracil.

22. A method for treating a subject having a cancer comprising
administering to the subject an effective amount of a pharmaceutical composition of claim 14 to treat the cancer.

23. The method of claim 22, wherein the cancer is leukemia (acute lymphocytic leukemia or chronic lymphocytic leukemia), multiple myeloma, lung cancer, head and neck cancer, endometrial cancer, ovarian cancer, cervical cancer, breast cancer, testicular cancer, prostate cancer, or Wilms' tumor.

24. A compound having a structure: [chemical structure]
